(19) United States
(12) Patent Application Publication
(10) Pub. No.: US 2020/0078330 A1
(43) Pub. Date: Mar. 12, 2020

(54) COMPOSITION FOR TREATING WOUNDS AND OTHER DERMATOLOGICAL CONDITIONS

(71) Applicant: SANVIO,INC., Athens, GA (US)

(72) Inventor: Tony GAY, Dublin, GA (US)

(21) Appl. No.: 16/468,229

(22) PCT Filed: Dec. 9, 2017
(86) PCT No.: PCT/US2017/065479

§ 371 Date: Jun. 10, 2019

Related U.S. Application Data
(60) Provisional application No. 62/431,839, filed on Dec. 9, 2016.

(51) Int. Cl. A61K 31/245; A61K 36/87; A61K 36/886; A61K 36/534; A61K 31/593; A61K 31/592; A61K 31/355; A61K 31/05; A61K 47/10; A61K 47/14; A61K 47/44

(52) U.S. Cl. CPC A61K 31/245; A61K 36/87; A61K 36/886; A61K 36/534; A61K 31/593; A61K 9/0014; A61K 31/355; A61K 31/05; A61K 47/10; A61K 47/14; A61K 47/44; A61K 31/592

(57) ABSTRACT

The present invention is directed to a composition that is suitable for the treatment of wounds and other dermatological conditions. The composition of the present invention comprises an anesthetic agent; an antipruritic agent; a mitotic agent; vitamin D or a derivative thereof; at least one of peppermint oil and thyme oil; one or more antioxidant agents; and a pharmaceutically acceptable carrier. The composition may further comprise one or more humectants and/or one or more emollients.

This application claims the benefit of U.S. Provisional Patent Application Ser. No. 62/431,839, filed Dec. 9, 2016, which is hereby incorporated by reference in its entirety.

### FIELD OF THE INVENTION

The present invention relates to compositions and methods that are suitable for the treatment of wounds and other dermatological conditions.

### BACKGROUND OF THE INVENTION

There are a plethora of dermatological conditions and wounds that plague individuals on a daily basis. These conditions include, without limitation, acute and chronic wounds, such as burn wounds and ulcers (e.g., diabetic ulcers, pressure ulcers, and dermal ulcers), and dermatological conditions such as blisters, rashes, eczema, dermatitis, psoriasis, viral infections (e.g., fever blisters), and bacterial infections and sores. There is also a plethora of topical anti-inflammatory agents, local anesthetics, and other pharmacological agents marketed to treat and/or alleviate each of these various conditions individually. However, a single composition that is effective for the treatment of a variety of these conditions does not exist. What is needed is a composition that is safe and effective for enhancing the healing of a variety of chronic and severe dermatological wounds and conditions. The composition and methods should be adaptable without regard to the type of wound, or the nature of the patient population, to which the subject belongs.

The present invention is directed towards overcoming these and other deficiencies in the art.

### SUMMARY OF THE INVENTION

A first aspect of the present invention is directed to a composition that comprises an anesthetic agent; an antipruritic agent; a mitotic agent; vitamin D or a derivative thereof; at least one of peppermint oil and thyme oil; one or more antioxidant agents; and a pharmaceutically acceptable carrier. In one embodiment, the composition further comprises one or more humectants and/or one or more emollients. In one embodiment, this composition does not contain a vasoconstrictive agent. In another embodiment, this composition does not contain an anti-fungal agent. In another embodiment, this composition does not contain a vasoconstrictive agent and does not contain an anti-fungal agent.

Another aspect of the present invention is directed to a composition that consists essentially of an anesthetic agent; an antipruritic agent; and one or more additional agents selected from a mitotic agent; vitamin D or a derivative thereof; one or more essential oils; one or more antioxidant agents; one or more humectants; one or more emollients; and a pharmaceutically acceptable carrier.

Another aspect of the present invention is directed to a wound dressing comprising a composition of the present invention as described herein and a wound dressing material.

Other aspects of the present invention are directed to methods of treating a wound or a dermatological condition that involve contacting the wound or dermatological condition with a composition of the present invention as described herein.

### DETAILED DESCRIPTION OF THE INVENTION

The present invention is generally directed to compositions and methods suitable for treating wounds and other dermatological conditions. Accordingly, a first aspect of the present invention is directed to a composition that comprises an anesthetic agent; an antipruritic agent; a mitotic agent; vitamin D or a derivative thereof; at least one of peppermint oil and thyme oil; one or more antioxidant agents; and a pharmaceutically acceptable carrier.

In accordance with this and all aspects of the present invention, a suitable anesthetic agent is any agent known in the art to control or reduce pain. In one embodiment, the anesthetic agent is a topical anesthetic agent. Suitable anesthetic agents are well known to those of skill in the art and include, for example and without limitation, benzocaine, lidocaine, chloroprocaine, mepivacaine, bupivacaine, articaine, etidocaine, levobupivacaine, tetracaine, prilocaine, ropivacaine, cocaine, oxyprocaine, hexylcaine, dibucaine, piperocaine, pramoxine, hydrocortisone, calamine, butamben, tetracaine, proxymetacaine, procaine and pharmaceutically acceptable acids, bases and salts thereof. The composition of the present invention may contain one, two, three, or more of the aforementioned anesthetic agents.

The anesthetic agent or agents may be present in the composition of the present invention in an amount from about 1% to about 50% of the total composition (w/w). The anesthetic agent may comprise about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40% , 45%, 50%, or any percent in between of the total composition. In one embodiment, the anesthetic agent comprises 5%-15% of the total composition. In one embodiment, the anesthetic agent comprises 8%-12% of the total compositions. In one embodiment, the anesthetic agent comprises 10% of the total composition. In one embodiment, the anesthetic agent comprises 15% of the total composition.

Antipruritic agents that are suitable for the composition of the present invention include topical steroids, including, but not limited to, clobetasol propionate, halobetasol propionate, augmented betamethasone dipropionate, diflorasone diacetate, betamethasone dipropionate, betamethasone valerate, fluocinonide, fluticasone propionate, mometasone furoate, desoximetasone, amcinonide, Topicort, hydrocortisone valerate, triamcinolone acetonide, alclometasone dipropionate, triamcinolone diacetate, desonide, hydrocortisone acetate.

In another embodiment, the antipruritic agent of the composition is a calcineurin inhibitor. Suitable calcineurin inhibitors include, without limitation, tacrolimus, pimecrolimus, and cyclosporine.

In another embodiment, the antipruritic agent of the composition is a topical antihistamine. Exemplary antihistamines include, without limitation, mepyramine maleate (pyrilamine), diphenhydramine, and doxepin.

In another embodiment, the antipruritic agent of the composition is a topical neuromodulatory agent. Suitable topical neuromodulatory agents include, without limitation, lidocaine, prilocaine, pramoxine, polidocanol, capsaicin, menthol, N-palmitoylethanolamine, naltrexone, and aprepitant.

In another embodiment, the antipruritic agent in the composition of the present invention is resorcinol or a derivative thereof. In another embodiment, the antipruritic agent in the composition of the present invention is crotamiton cream. In another embodiment, the antipruritic agent in the composition of the present invention is chlorphenesin. In another embodiment, the antipruritic agent in the composition of the present invention is menthol or menthol/camphor. In another embodiment, the antipruritic agent in the composition is calamine (i.e., zinc oxide and ferric oxide).

The composition of the present invention may contain one, two, three, or more of the aforementioned antipruritic agents. The antipruritic agent may be present in the composition of the present invention in an amount from about 1% to about 50% of the total composition. The antipruritic agent may comprise about 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10%, 15%, 20%, 25%, 30%, 35%, 40%, 45%, 50%, or any percent in between of the total composition. In one embodiment, the antipruritic agent comprises 0.5%-2.5% of the total composition. In one embodiment, the antipruritic agent comprises 1.0% -2.0% of the total composition. In one embodiment, the antipruritic agent comprises 1.5% of the total composition.

The composition of the present invention also comprises a mitotic agent or an agent that antagonizes decreased cell growth. In one embodiment, this agent is a retinoid (i.e., a vitamin A derivative) or derivative thereof. Suitable derivatives of retinoid include, without limitation, retinol, retinal, retinoic acid, retinyl acetate, retinyl palmitate, retinyl ascorbate, acitretin, isotretinoin, adapalene and tazarotene. The composition of the present invention may contain one, two, three, or more of the aforementioned mitotic agents.

The mitotic agent may be present in the composition of the present invention in an amount from about 0.01% to about 10% of the total composition. The mitotic agent may comprise about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, or any percent in between of the total composition. In one embodiment, the mitotic agent comprises 0.01%-5% of the total composition. In one embodiment, the mitotic agent comprises 0.05%-1% of the total compositions. In one embodiment, the mitotic agent comprises 0.1% of the total composition.

The composition of the present invention also contains one or more antioxidant agents. Suitable antioxidant agents include, without limitation, vitamin E and derivatives thereof (e.g., α-tocopherol, β-tocopherol, γ-tocopherol, δ-tocopherol, tocopherol acetate, tocotrienol), ascorbic acid and ascorbic acid salts, ascorbyl esters of fatty acids, ascorbic acid derivatives, butylated hydroxy benzoic acids, 6-hydroxy-2,5,7,8-tetramethylchroman-2-carboxylic acid, gallic acid and gallic acid alkyl esters, uric acid, uric acid salts and alkyl esters, sorbic acid and sorbic acid salts, lipoic acid, amines, sulfhydryl compounds, dihydroxy fumaric acid, dihydroxy fumaric acid salts, nordihydroguaiaretic acid, bioflavonoids, curcumin, lysine, methionine, proline, superoxide dismutase, silymarin, tea extracts, grape skin/seed extracts, melanin, aloe leaf extract, thyme flower leaf oil, and rosemary extracts.

The one or more antioxidant agents may be present in the composition of the present invention in an amount from about 0.01% to about 10% of the total composition. The antioxidant agent(s) may comprise about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, or any percent in between of the total composition. In one embodiment, the one or more antioxidant agents comprise 0.01%-5% of the total composition. In one embodiment, the one or more antioxidant agents comprise 0.05%-1% of the total composition. In one embodiment, the one or more antioxidant agents comprise 0.4%-0.5% of the total composition.

The composition of the present invention also contains vitamin D or a derivative thereof. Exemplary vitamin D compounds include vitamin D₃, also known as cholecalciferol, and vitamin D₂, also known as ergocalciferol. Exemplary vitamine D₃ analogs such as calcipotriol, tacalcitol, maxacalcitol, and calcitriol are also suitable for the composition of the present invention. The vitamin D or a derivative thereof may be present in the composition of the present invention in an amount from 0.01% to about 10% of the total composition. The vitamin D or a derivative thereof may comprise about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, or any percent in between of the total composition. In one embodiment, the vitamin D or a derivative thereof comprises 0.01%-5% of the total composition. In one embodiment, the vitamin D or a derivative thereof comprises 0.05%-1% of the total composition. In one embodiment, the vitamin D or a derivative thereof comprises 0.1% of the total composition.

The composition of the present invention contains one or more essential oils. In one embodiment, the composition contains peppermint (Mentha piperita) oil, thyme (Thymus vulgaris) flower leaf oil, or a combination of peppermint and thyme flower leaf oil. Other suitable essential oils that can be included in the composition of the present invention include, without limitation, basil oil, camphor oil, cardamom oil, carrot oil, citronella oil, clary sage oil, clove oil, cypress oil, frankincense oil, ginger oil, grapefruit oil, hyssop oil, jasmine oil, lavender oil, lemon oil, mandarin oil, marjoram oil, myrrh oil, neroli oil, nutmeg oil, petitgrain oil, sage oil, tangerine oil, vanilla oil, verbena oil, as well as any other therapeutically beneficial oil know in the art of herbal medication.

The one or more essential oils may be present in the composition of the present invention in an amount from about 0.01% to about 10% of the total composition. The one or more essential oils may comprise about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, or any percent in between of the total composition. In one embodiment, the one or more essential oils comprise 0.01%-5% of the total composition. In one embodiment, the one or more essential oils comprise 0.05%-1% of the total composition. In one embodiment, the one or more essential oils comprise 0.1%-0.75% of the total composition. In one embodiment, the one or more essential oils comprise 0.33% of the total composition. In one embodiment, the one or more essential oils comprise 0.66% of the total composition.

In one embodiment, the composition of the present invention also contains one or more humectants. As used herein, a “humectant” is a substance that helps retain moisture and also prevents evaporation. Suitable humectants can be synthetic or natural, and include without limitation, beeswax, glycerin, glyceryl triacetate, glycerol, aloe leaf extract, honey, seaweed, sorbitol, xylitol, maltitol, polydextrose, urea, butylene glycol, hexylene glycol, caprylyl glycol, propylene glycol and propylene glycol derivatives, tetraglycol, lactic acid, 1,4 dihydroxyhexane, 1,2,6-hexane triol, hyaluronic acid lactamide monoethanolamine, acetamide monoethanolamine, glycolic acid, polyethylene glycol, silicone, tremella extract, dicyanamide, sodium PCA, sodium lactate, and alpha and beta hydroxy acids.

The one or more humectants may be present in the composition of the present invention in an amount from about 0.01% to about 10% of the total composition. The one or more humectants may comprise about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, or any percent in between of the total composition. In one embodiment, the one or more humectants comprise 0.1%-7% of the total composition. In one embodiment, the one or more humectants comprise 0.3%-6% of the total composition. In one embodiment, the one or more humectants comprise 0.5%-6% of the total composition. In one embodiment, the one or more humectants comprise 0.5% of the total composition. In one embodiment, the one or more humectants comprise 5% of the total composition. In one embodiment, the one or more humectants comprise 5%-6% of the total composition.

In one embodiment of the present invention, the composition as described herein also contains one or more emollients. As used herein, an emollient is an agent that reduces water loss from the skin or tissue being treated. Suitable emollients include, without limitation, natural oil, plant-derived oil, mineral oil, silicone oil (e.g., dimethyl silicone, polysiloxane, polydimethylsiloxane, and mixtures thereof), polyunsaturated fatty acid, paraffin, beeswax, squalene, cetyl oil, petrolatum, and lanolin and its derivatives. Exemplary emollients also include glyceryl monostearate, isopropyl myristate, isopropyl palmitate, isopropyl isostearate, diisopropyl adipate, diisopropyl dimerate, maleated soybean oil, octyl palmitate, cetyl lactate, cetyl ricinoleate, tocopheryl acetate, cetyl acetate, tocopheryl linoleate, wheat germ glycerides, arachidyl propionate, myristyl lactate, decyl oleate, propylene glycol, propylene glycol ricinoleate, isopropyl lanolate, pentaerythrityl tetrastearate, neopentylglycol dicaprylate/dicaprate, caprylyl glycol, isononyl isononanoate, isotridecyl isononanoate, myristyl myristate, octyl dodecanol, sucrose esters of fatty acids and octyl hydroxystearate.

The one or more emollients may be present in the composition of the present invention in an amount from about 1% to about 50% of the total composition (w/w). The one or more emollients may comprise about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40% , 45%, 50%, or any percent in between of the total composition. In one embodiment, the one or more emollients comprise 1%-20% of the total composition. In one embodiment, the one or more emollients comprise 2%-18% of the total compositions. In one embodiment, the one or more emollients comprise 5%-16% of the total composition. In one embodiment, the one or more emollients comprise 16.0%-16.5%. In one embodiment, the one or more emollients comprise 5%-7%.

The composition of the present invention may further include other healing agents, such as anti-inflammatory agents, antibiotic agents, antiseptic agents, healing promoters, and combinations thereof.

Anti-inflammatory agents useful for dispersion in the composition of the present invention include, without limitation, analgesics (e.g., NSAIDS and salicylates), hormones (glucocorticoids), and skin and mucous membrane agents. Specifically, the anti-inflammatory agent can include dexamethasone. Alternatively, the anti-inflammatory agent can include sirolimus (rapamycin). The anti-inflammatory agents may comprise any suitable concentration in the composition that is effective to induce or to enhance the desired wound healing. Preferably, the lowest effective concentration that can contribute to the desired wound healing is utilized.

A variety of antibiotics can also be dispersed in the composition of the present invention to indirectly promote natural healing processes by preventing or controlling infection. Suitable antibiotics include aminoglycoside antibiotics (e.g., gentamycin, tobramycin), quinolones (e.g., ciprofloxacin), beta-lactams (e.g., ampicillin, cephalosporins), erythromycin, vancomycin, oxacillin, cloxacillin, methicillin, lincomycin, silver sulfadiazine, and colistin. In one embodiment, the antibiotic is silver sulfadiazine. The concentration of the antibiotic within the composition of the present invention is the concentration effective to induce or to enhance the desired healing effect. Preferably, the lowest effective concentration that can contribute to the desired healing effect is utilized.

The composition may further include one or more antiseptic agents. Suitable antiseptic agents include, without limitation, triclosan, phenoxy isopropanol, chlorhexidine gluconate, povidone iodine, or any combination thereof.

The composition may further include one or more wound healing promoters. Suitable wound healing promoters include, without limitation, vitamin A and synthetic inhibitors of lipid peroxidation. Other suitable wound healing promoters include agents that promote natural wound healing processes by endothelial cells. These wound healing agents include any bioactive agent that donates, transfers, or releases nitric oxide, elevates endogenous levels of nitric oxide, stimulates endogenous synthesis of nitric oxide, or serves as a substrate for nitric oxide synthase or that inhibits proliferation of smooth muscle cells. Such wound-healing agents include, for example, aminoxyls, furoxans, nitrosothiols, nitrates and anthocyanins; nucleosides, such as adenosine; nucleotides, such as adenosine diphosphate (ADP) and adenosine triphosphate (ATP); histamine and catecholamines; lipid molecules, such as sphingosine-1-phosphate and lysophosphatidic acid; amino acids, such as arginine and lysine; peptides such as the bradykinins, substance P and calcium gene-related peptide (CGRP), and proteins, such as insulin, vascular endothelial growth factor (VEGF), and thrombin. The concentration of the wound healing promoter within the composition of the present invention is the concentration effective to induce or to enhance the desired healing effect. Preferably, the lowest effective concentration that can contribute to the desired healing effect is utilized.

In one embodiment of the present invention, the composition described herein does not contain a vasoconstrictor or vasopressor agent, such as phenylephrine or the like. As used herein, a vasoconstrictor agent is an agent that narrows or constricts blood vessels. In another embodiment of the present invention, the composition as described herein does not contain an anti-fungal agent, such as methylparaben or the like. In another embodiment of the present invention, the composition described herein does not contain a vasoconstrictor agent and does not contain an anti-fungal agent.

In one embodiment, the composition of the present invention consists essentially of an anesthetic agent; an antipruritic agent; and one or more additional agents selected from a mitotic agent; vitamin D or a derivative thereof; one or more essential oils; one or more antioxidant agents, one or more humectants, one or more emollients, and a pharmaceutically acceptable carrier.

In one embodiment, the composition of the present invention consists essentially of an anesthetic agent; an antipruritic agent; a mitotic agent; vitamin D or a derivative thereof; one or more essential oils; one or more antioxidant agents, and a pharmaceutically acceptable carrier.

In another embodiment, the composition of the present invention consists essentially of an anesthetic agent; an antipruritic agent; a mitotic agent; one or more humectants; one or more emollients; vitamin D or a derivative thereof; one or more essential oils; one or more antioxidant agents, and a pharmaceutically acceptable carrier.

As used herein, the phrase “consists essentially of” means that the recited composition contains the recited components without containing any other components that materially affect the basic and novel characteristic(s) of the composition, e.g., does not contain other active components having wound healing properties. Accordingly, the aforementioned compositions may further comprise one or more preservatives, binders, fillers, carriers, stabilizing agents, emulsifiers, surfactants, diluents, excipients, and/or buffering agents as described herein that maintain or enhance the stability and shelf-life of a particular formulation of the composition.

An exemplary composition of the present invention comprises benzocaine or a derivative thereof, resorcinol, a retinoid, vitamin D or a derivative thereof, vitamin E or a derivative thereof, peppermint oil, thyme flower leaf oil, and aloe leaf extract. In one embodiment, the retinoid is retinyl palmitate, vitamin D is cholecalciferol, and vitamin E is tocopherol. In one embodiment, the exemplary composition further comprises one or more humectants, such as caprylyl glycol, hexylene glycol, and/or propylene glycol, and one or more emollients, such as isopropyl palmitate, mineral oil, and/or glyceryl monostearate. In one embodiment, this exemplary composition does not contain a vasoconstrictor agent and/or does not contain an anti-fungal agent. In accordance with this embodiment, the composition may further comprise one or more preservatives, binders, fillers, carriers, stabilizing agents, emulsifiers, surfactants, diluents, excipients, and/or buffering agents as described herein that maintain or enhance the stability and shelf-life of a particular formulation of the composition.

In another embodiment, the composition of the present invention consists essentially of benzocaine or a derivative thereof, resorcinol, a retinoid, vitamin D or a derivative thereof, vitamin E or a derivative thereof, peppermint oil, thyme flower leaf oil, and aloe leaf extract. In one embodiment, the retinoid is retinyl palmitate, vitamin D is cholecalciferol, and vitamin E is tocopherol. In one embodiment, this exemplary composition does not contain a vasoconstrictor agent and/or does not contain an anti-fungal agent. In accordance with this embodiment, the composition may further comprise one or more preservatives, binders, fillers, carriers, stabilizing agents, emulsifiers, surfactants, diluents, excipients, and/or buffering agents as described herein that maintain or enhance the stability and shelf-life of a particular formulation of the composition.

In another embodiment, this exemplary composition consists essentially of benzocaine or a derivative thereof, resorcinol, a retinoid, vitamin D or a derivative thereof, vitamin E or a derivative thereof, peppermint oil, thyme flower leaf oil, aloe leaf extract, one or more humectant selected from caprylyl glycol, hexylene glycol, and/or propylene glycol, and one or more emollients selected from isopropyl palmitate, mineral oil, and/or glyceryl monostearate. In one embodiment, the retinoid is retinyl palmitate, vitamin D is cholecalciferol, and vitamin E is tocopherol. In one embodiment, this exemplary composition does not contain a vasoconstrictor agent and/or does not contain an anti-fungal agent. In accordance with this embodiment, the composition may further comprise one or more preservatives, binders, fillers, carriers, stabilizing agents, emulsifiers, surfactants, diluents, excipients, and/or buffering agents as described herein that maintain or enhance the stability and shelf-life of a particular formulation of the composition.

In another embodiment, this exemplary composition consists essentially of 5%-15% benzocaine or a derivative thereof, 1%-2% resorcinol, 0.05%-1.0% retinoid, 0.05%-1.0% vitamin D or a derivative thereof, 0.05%-1.0% vitamin E or a derivative thereof, 0.1%-1.0% peppermint oil, 0.1%-1.0% thyme flower leaf oil, 0.001%-0.1% aloe leaf extract. In one embodiment, the composition further contains 0.5%-5.0% humectant such as caprylyl glycol, hexylene glycol, and/or propylene glycol. In one embodiment, the composition further contains 1%-20% emollient such as isopropyl palmitate, mineral oil, and/or glyceryl monostearate. In accordance with this embodiment, the retinoid is retinyl palmitate, vitamin D is cholecalciferol, and vitamin E is tocopherol. In accordance with this embodiment, the composition may further comprise one or more components as described herein suitable for enhancing the stability and shelf-life of the composition.

In another embodiment, this exemplary composition consists essentially of 15% benzocaine or a derivative thereof, 1.5% resorcinol, 0.1% retinoid, 0.1% vitamin D or a derivative thereof, 0.1% vitamin E or a derivative thereof, 0.33% peppermint oil, 0.33% thyme flower leaf oil, 0.01% aloe leaf extract. In one embodiment, the composition further contains a humectant such as caprylyl glycol (0.3%) and/or propylene glycol (5%). In one embodiment, the composition further contains an emollient such as isopropyl palmitate (2.5%), mineral oil (10%), and/or glyceryl monostearate (3.5%). In accordance with this embodiment, the retinoid is retinyl palmitate, vitamin D is cholecalciferol, and vitamin E is tocopherol. In accordance with this embodiment, the composition may further comprise a pharmaceutically acceptable carrier and one or more components as described herein suitable for enhancing the stability and shelf-life of the composition.

In another embodiment, an exemplary composition of the present invention comprises benzocaine or a derivative thereof, resorcinol, a retinoid, lanolin, beeswax, petrolatum, glycerin, vitamin D or a derivative thereof, vitamin E or a derivative thereof, peppermint oil, thyme oil; and aloe leaf extract. In one embodiment, the retinoid is retinyl palmitate, vitamin D is cholecalciferol, and vitamin E is tocopherol. In one embodiment, this exemplary composition does not contain a vasoconstrictor agent and/or does not contain an anti-fungal agent. In accordance with this embodiment, the composition may further comprise one or more components as described herein suitable for enhancing the stability and shelf-life of the composition.

In another embodiment of the present invention, the composition described herein consists essentially of benzocaine or a derivative thereof, resorcinol, a retinoid, lanolin, beeswax, petrolatum, glycerin, vitamin D or a derivative thereof, vitamin E or a derivative thereof, peppermint oil, thyme oil, and aloe leaf extract. In one embodiment, the retinoid is retinyl palmitate, vitamin D is cholecalciferol, and vitamin E is tocopherol. In one embodiment, this exemplary composition does not contain a vasoconstrictor agent and/or does not contain an anti-fungal agent. In accordance with this embodiment, the composition may further comprise one or more components as described herein suitable for enhancing the stability and shelf-life of the composition.

These compositions of the present invention are suitable for administration to mammals for veterinary use, such as with domestic and small animals (dogs, cats, rodents, horses, etc.), livestock (cows, pigs, poultry), and for clinical use in humans in a manner similar to other therapeutic agents. In general, the dosage required for therapeutic efficacy will vary according to the type of use and mode of administration, as well as the particularized requirements of individual and condition being treated.

In one embodiment, the composition of the present invention is formulated for topical or transdermal administration to the skin or mucosa and can take the form of an ointment, gel, lotion, cream, powder, paste, emulsion, suspension, spray-solution, spray-on gel, foam, aerosol, or any other formulation that is suitable for topical or transdermal administration. The composition can also be formulated to include a delivery vehicle that is suitable for topical or transdermal delivery such as liposomes, ethosomes, microcapsules, microspheres, or the like.

Such compositions are typically prepared as liquid solutions or suspensions, or in solid forms. Formulations for wound healing usually will include such normally employed additives such as binders, fillers, carriers, preservatives, stabilizing agents, emulsifiers, buffers and excipients such as, for example, pharmaceutical grades of mannitol, lactose, starch, magnesium stearate, sodium saccharin, cellulose, magnesium carbonate, polyalkylene glycols, triglycerides, stearic acid, triethanolamine, acrylates/C10-30 alkyl acrylate crosspolymer, isopropyl palmitate, cetyl alcohol, phenoxyethanol, ethylhexyglycerin, edetate disodium, and the like. The composition of the present invention may also be mixed with diluents or excipients which are physiologically tolerable and compatible. Suitable diluents and excipients are, for example, purified water, saline, dextrose, glycerol, or the like, and combinations thereof. In addition, if desired the compositions may contain minor amounts of auxiliary substances such as wetting or emulsifying agents, surfactants, stabilizing or pH buffering agents, including, and without limitation, triethanolamine, acrylates/C10-30 alkyl acrylate crosspolymer, PEG-100 stearate, hexylene glycol, cetyl alcohol, glyceryl monostearate, stearic acid.

The composition of the present invention may comprise one or more components to provide structure and strength to the desired formulation. Suitable components for providing structure and strength of the composition include, without limitation gum karaya, a polyacrylamide, xanthum gum, guar gum, a natural polymer, a synthetic polymer, a hydrophilic polymer, a hydrocolloidal polymer, starch, a starch derivative, vinyl acetate copolymer, polyvinyl pyrrolidone, polyethylene oxide, algin, derivatives of algin, a polyacrylate, polymaleic acid, polymaleic anhydride, a polyurethane, a polyurea, gum acacia, locust bean gum, modified guar gum, maltodextrin, carboxymethyl cellulose, carboxypropyl cellulose, polyvinyl alcohol, poly AMPS, acrylates/C10-30 alkyl acrylate crosspolymer, hexylene glycol, edetate disodium, or a mixture thereof.

When formulating the composition of the present invention in a gel form, such composition may include one or more gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate. The preferred concentration of the gelling agent may range from 0.1 to 4 percent by weight of the total composition

Another aspect of the present invention is directed to a wound-dressing comprising the composition as described supra and a wound dressing material.

The wound dressing material can be any material applied to a wound for protection, absorbance, drainage, etc. Numerous types of dressings are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), cellophane (cellulose with a plasticizer), gauze, alginate, polysaccharide paste, granules, and beads.

Another aspect of the present invention is directed to a method of treating a wound that involves providing the composition of the present invention or a wound dressing containing the composition described herein, and applying the composition or the wound dressing containing the composition to the wound under conditions suitable to promote healing of the wound. Preferable, administration of the composition or wound healing dressing enhances or increases the rate of healing of the wound being treated.

In accordance with this aspect of the present invention, the composition of the present invention is administered in a suitable dosage that is effective to induce or to enhance the desired wound healing. Preferably, the lowest effective dose that can contribute to the desired wound healing is utilized. The wound healing composition of the present invention is preferably reapplied periodically until the wound heals. For example, the wound healing composition may be applied twice daily, daily, every other day, every two days, every three days, etc., until the desired healing is achieved (e.g., 50-70% wound closure). The optimal dosage and frequency of application will vary depending on the wound and can be determined using techniques readily known to one of skill in the art.

Depending on the type of wound and formulation, the composition of the present invention can be applied to the wound by spraying or misting a solution or suspension onto the region of the wound, or spreading the lotion, cream, gel, emulsion, ointment, foam, mucoadhesive, or paste containing the composition onto the wound or region thereof.

The wound healing composition of the present invention is suitable for treatment of internal and external wounds. The wound healing composition of the present invention is particularly suitable for treatment of chronic non-healing wounds, such as diabetic ulcers, pressure ulcers, leg ulcers, dermal ulcers, burns, corneal wounds, and incisions which involve body tissues being cut, abraded, or otherwise damages.

In one embodiment of the present invention, the wound to be treated is a burn wound. Burn wounds are tissue injuries that result from exposure to heat, chemicals, sunlight, electricity, radiation, etc. Burns caused by heat, or thermal burns, are the most common. Chemical burns resemble thermal burns. Though burn wounds tend to occur most often on the skin, other body structures may be affected. For example, a severe burn may penetrate down to the fat, muscle or bone.

Treating a burn using the composition and methods described herein involves reducing the extent of tissue destruction and necrosis caused by the burn injury. In another embodiment, treating a burn using the composition and methods described herein involves enhancing one or more aspects of the repair process and reducing the overall healing time. The composition of the present invention contributes to and/or enhances the activity of one or more phases of wound healing, e.g., the inflammatory phase, the proliferative phase, and the remodeling phase to decrease healing time while improving the structure and integrity of the repaired tissue. In another embodiment treating a burn injury with the composition and methods of the present invention decreases the pain, scarring, and/or hyperpigmentation produced by the burn injury.

Another aspect of the present invention is directed to a method of treating a dermatological condition of a subject that involves selecting a subject having a dermatological condition and contacting the dermatological condition of the subject with the composition described herein in an amount effective to promote healing of said dermatological condition.

“Treating” a dermatological condition in accordance with this embodiment of the present invention involves reducing, alleviating, or ameliorating one or more symptoms of the condition. Treating the dermatological condition may alternatively involve delaying the onset or worsening of one or more symptoms of the condition. For example, the method disclosed herein is considered to be a treatment if there is about a 5% reduction in one or more symptoms of the condition in a subject when compared to the subject prior to the treatment or when compared to a control subject. Thus reduction in one or more symptoms can be about 5%, 10%, 20%, 30%, 40%, 50%, 60%, 70%, 80%, 90%, 100% or any amount of reduction in between.

Dermatological conditions that are suitable for treatment with the composition described herein include, without limitation burns, fever blisters, ingrown hairs, eczema, bed sores, cuts, bites, shingles, and rash, dermatitis including contact dermatitis, atopic dermatitis, seborrheic dermatitis, nummular dermatitis, chronic dermatitis of the hands and feet, generalized exfoliative dermatitis, stasis dermatitis; lichen simplex chronicus; diaper rash; bacterial infections including cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma; viral infections; disorders of hair follicles and sebaceous glands including acne, rosacea, perioral dermatitis, pseudofolliculitis barbae, keratinous cyst; scaling papular diseases including psoriasis, pityriasis rosea, lichen planus, pityriasis rubra pilaris; benign tumors including moles, dysplastic nevi, skin tags, lipomas, angiomas, pyogenic granuloma, seborrheic keratoses, dermatofibroma, keratoacanthoma, keloid; sunburn, chronic effects of sunlight, photosensitivity; bullous diseases including pemphigus, bullous pemphigoid, dermatitis herpetiformis, linear immunoglobulin A disease; pigmentation disorders including hypopigmentation such as vitiligo, albinism and postinflammatory hypopigmentation and hyperpigmentation such as melasma (chloasma), drug-induced hyperpigmentation, postinflammatory hyperpigmentation; disorders of cornification including ichthyosis, keratosis pilaris, calluses and corns, actinic keratosis; pressure sores; inflammatory skin reactions including, erythema multiforme, erythema nodosum, and granuloma annulare.

Depending on the dermatological condition being treated, the composition of the present invention can be applied to the wound by spraying or misting a solution or suspension onto region of skin or tissue affected by the condition, or spreading the lotion, cream, gel, emulsion, ointment, foam, mucoadhesive, or paste containing the composition onto the skin or tissue affected by the condition. The application of the composition to the dermatological condition is preferably reapplied periodically until the desired reduction of one or more symptoms or amelioration of the condition is achieved. For example, the composition of the present invention may be applied twice daily, daily, every other day, every two days, every three days, etc., until the desired level of treatment is achieved. The optimal dosage and frequency of application will vary depending on the dermatological condition being treated and can be determined using techniques readily known to one of skill in the art.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A composition comprising:
an anesthetic agent;
an antipruritic agent;
a mitotic agent;
vitamin D or a derivative thereof;
at least one of peppermint oil and thyme flower leaf oil;
one or more antioxidant agents; and
a pharmaceutically acceptable carrier.

2. The composition of claim 1, wherein the anesthetic agent is selected from the group consisting of benzocaine, lidocaine, chloroprocaine, mepivacaine, bupivacaine, articaine, etidocaine, levobupivacaine, tetracaine, prilocaine, ropivacaine, cocaine, oxyprocaine, hexylcaine, dibucaine, piperocaine, pramoxine, hydrocortisone, calamine, butamben, tetracaine, proxymetacaine, procaine and pharmaceutically acceptable acids, bases and salts thereof.

3. The composition of claim 1, wherein the anesthetic agent is benzocaine.

4. The composition of claim 1, wherein the antipruritic agent is resorcinol, crotaminton cream, chlorphenesin, or menthol.

5. The composition of claim 1, wherein the antipruritic agent is selected from the group consisting of a topical corticosteroid, a calcineurin inhibitor, a topical antihistamine, and a topical neuromodulator.

6. The composition of claim 1, wherein the mitotic agent is a retinoid or derivative thereof selected from the group consisting of retinol, retinal, retinoic acid, retinyl acetate, retinyl palmitate, retinyl ascorbate, acitretin, isotretinoin, adapalene and tazarotene.

7. The composition of claim 1, wherein the one or more antioxidant agents is selected from the group consisting of vitamin E and derivatives thereof, ascorbic acid and ascorbic acid salts, ascorbyl esters of fatty acids, ascorbic acid derivatives, butylated hydroxy benzoic acids, 6-hydroxy-2,5, 7,8-tetramethylchroman-2-carboxylic acid, gallic acid and gallic acid alkyl esters, uric acid, uric acid salts and alkyl esters, sorbic acid and sorbic acid salts, lipoic acid, amines, sulfhydryl compounds, dihydroxy fumaric acid, dihydroxy fumaric acid salts, nordihydroguaiaretic acid, bioflavonoids, curcumin, lysine, methionine, proline, superoxide dismutase, silymarin, tea extracts, grape skin/seed extracts, melanin, aloe leaf extract, and rosemary extracts.

8. The composition of claim 1, wherein the composition contains peppermint oil and thyme flower leaf oil.

9. The composition of claim 1 further comprising one or more humectants.

10. The composition of claim 9, wherein the one or more humectants is selected from the group consisting of beeswax, glycerin, glyceryl triacetate, glycerol, aloe leaf extract, honey, seaweed, sorbitol, xylitol, maltitol, polydextrose, urea, butylene glycol, hexylene glycol, propylene glycol, tetraglycol, lactic acid, 1,4 dihydroxyhexane, 1,2,6-hexane triol, hyaluronic acid lactamide monoethanolamine, acetamide monoethanolamine, glycolic acid, caprylyl glycol, hexylene glycol, polyethylene glycol, silicone, tremella extract, dicyanamide, sodium PCA, sodium lactate, and alpha and beta hydroxy acids.

11. The composition of claim 1 further comprising one or more emollients.

12. The composition of claim 11, wherein the one or more emollients is selected from the group consisting of a natural oil, plant-derived oil, mineral oil, isopropyl palmitate, glyceryl monostearate, silicone oil, polyunsaturated fatty acid, paraffin, beeswax, squalene, cetyl oil, petrolatum, and lanolin and its derivatives.

13. The composition of claim 1 comprising:
benzocaine or a derivative thereof;
resorcinol;
a retinoid;
vitamin D or a derivative thereof;
vitamin E or a derivative thereof;
peppermint oil;
thyme flower leaf oil;
aloe leaf extract; and
a pharmaceutically acceptable carrier.

14. The composition of claim 13 further comprising:
caprylyl glycol, propylene glycol, or a combination thereof.

15. The composition of claim 13 further comprising:
glyceryl monostearate, mineral oil, isopropyl palmitate, or any combination thereof.

16. The composition of claim 1 consisting essentially of:
benzocaine or a derivative thereof;
resorcinol;
a retinoid;
vitamin D or a derivative thereof;
vitamin E or a derivative thereof;
peppermint oil;
thyme flower leaf oil;
aloe leaf extract; and
a pharmaceutically acceptable carrier.

17. The composition of claim 1, wherein said composition does not contain a vasoconstrictor agent.

18. The composition of claim 1, wherein said composition does not contain an anti-fungal agent.

19. A composition consisting essentially of:
an anesthetic agent;
an antipruritic agent; and one or more additional agents selected from:
a mitotic agent;
one or more humectants;
one or more emollients;
vitamin D or a derivative thereof;
one or more essential oils;
one or more antioxidant agents; and
a pharmaceutically acceptable carrier.

20. The composition of claim 1, wherein said composition is formulated for topical administration.

21. The composition of claim 20, wherein said composition is in the form of an ointment, gel, lotion, cream, powder, paste, spray-solution, spray-on gel, foam, or aerosol.

22. A wound dressing comprising:
the composition of claim 1; and
a wound dressing material.

23. The wound dressing of claim 22, wherein the wound dressing material is selected from the group consisting of gauze, film, gel, hydrocolloid, alginate, hydrogel, polysaccharide paste, granules, and beads.

24. A method of treating a wound comprising:
providing the composition of claim 1, and
applying said composition or said wound dressing to the wound under conditions suitable to promote healing of the wound.

25. A method of treating a dermatological condition, said method comprising:
selecting a subject having a dermatological condition and
contacting the dermatological condition of the subject with the composition of claim 1 in an amount effective to promote healing of said dermatological condition.

26. The method of claim 25, wherein the dermatological condition is selected from the group consisting of a burn, fever blister, ingrown hair, psoriasis, eczema, dermatitis, bed sores, shingles, and rash.

27. The method of claim 25 further comprising: repeating said contacting.
